(19) United States
(12) Patent Application Publication
(10) Pub. No.: US 2017/0307598 A1
(43) Pub. Date: Oct. 26, 2017

(54) TISSUE-MIMICKING HYDROGEL COMPOSITIONS FOR BIOFABRICATION

(71) Applicant: Wake Forest University Health Sciences, Winston-Salem, NC (US)

(72) Inventors: Aleksander Skardal, Winston-Salem, NC (US); Shay Soker, Greensboro, NC (US)

(21) Appl. No.: 15/520,766

(22) PCT Filed: Oct. 15, 2015
(86) PCT No.: PCT/US15/55699

§ 371 Date: Apr. 20, 2017

Related U.S. Application Data
(60) Provisional application No. 62/068,218, filed on Oct. 24, 2014.

(51) Int. Cl. G01N 33/50; B29C 64/106; C12N 5/071; B33Y 70/00; B01L 3/00; B33Y 10/00; B33Y 80/00; B29L 31/40

(52) U.S. Cl. CPC G01N 33/5082; B33Y 70/00; G01N 33/5014; B29C 64/106; B01L 3/5027; C12N 5/0671; B29K 2089/00; B29K 2005/00; B01L 2300/1894; B01L 2200/12; B33Y 80/00; B29K 2995/0082; B29L 2031/40; C12N 2513/00; C12N 2533/30; B33Y 10/00; C12N 2537/10

(57) ABSTRACT

An extrudable hydrogel composition useful for making a three-dimensional organ construct includes a cross-linkable prepolymer, a post-deposition crosslinking group, optionally, an initiator that catalyzes the reaction between the prepolymer and said the crosslinking group; live cells (e.g., plant, animal, or microbial cells), optionally at least one one growth factor, and optionally water to balance. Methods of using the same and products so made are also described.

### RELATED APPLICATION

This application claims the benefit of U.S. Provisional Patent Application Ser. No.62/068,218, filed Oct. 24, 2014, the disclosure of which is incorporated by reference herein in its entirety.

### GOVERNMENT SUPPORT

This invention was made with government support under Contract No. N6601-13-C-2027 awarded by the Defense Threat Reduction Agency. The US Government has certain rights to this invention.

### FIELD OF THE INVENTION

The present invention concerns hydrogel “bioink” compositions useful for fabrication of artificial tissue constructs, methods of using the same, and products foamed therefrom.

### BACKGROUND OF THE INVENTION

Biofabrication technologies have emerged as tissue engineering approaches for building organs and organoids or tissue constructs. The combination of biocompatible materials and rapid prototyping makes provides a way to address the intricacies needed in viable tissues. One of the hurdles associated with biofabrication is the interfacing between the deposition/fabrication hardware and different types of biomaterials (or “bio-inks”) being deposited. Standard hydrogels pose design problems because they are either printed as fluid solutions, limiting mechanical properties, or printed as solid hydrogels and broken up upon the extrusion process.

### SUMMARY OF THE INVENTION

Embodiments of the materials described herein address the issues noted above by being extrudable, and by possessing a post-deposition or secondary crosslinking step which stabilizes and increases the stiffness of the end product to match a range of tissue types. Additionally, these “bioink” compositions can be supplemented with biochemical factors derived from tissues that result in a biochemical environment more like that of an in vivo tissue that cells in the biofabricated constructs then experience. These factors—both biochemical and mechanical—can increase the ability to maintain viable cells in culture and to increase their functionality for the duration of culture.

In view of the foregoing, the present invention provides an extrudable hydrogel composition (or “bioink”) useful for making a three-dimensional organ construct. The composition comprises:

(a) a cross-linkable prepolymer;

(b) a post-deposition crosslinking group (also referred to as a second crosslinking group);

(c) optionally, but in some embodiments preferably, an initiator that catalyzes the reaction between said prepolymer and said post-deposition crosslinking group;

(d) live cells (e.g., live animal cells);

(e) optionally, but in some embodiments preferably, at least one growth factor; and

(f) optionally, water to balance. Methods of using the foregoing, and products produced therefrom, are also described herein.

Some embodiments of the invention provide advantages as follows:

Control over biochemical properties. Tissues in the body have complex compositions: Various subpopulations of cells secrete signaling molecules such as growth factors and other cytokines that aid in maintaining viability and function of cells in tissues. Extracellular matrix is comprised of proteins and polymers that provide structure to the tissue and also interact with cell receptors acting as another type of signaling. Additionally, some ECM components bind growth factors (heparin, heparan sulfate) and slowly release them to the cells over time. The combination of these signals varies from tissue to tissue. We previously developed a method for providing components specific to the liver within a hydrogel in order to support primary human hepatocytes. By decellularizing any tissue, pulverizing it, dissolving it, we can produce tissue-specific biochemical signals from any tissue to cells in 3-D hydrogel constructs.

Control over mechanical properties. Mechanical properties, specifically elastic modulus, are important for 2 major reasons. First, as has been described in earlier reports, control over the hydrogel bioink stiffness allows for extrusion-based biofabrication using a soft gel, which can then be stiffened afterwards by secondary crosslinking to increase stability. Second, this second crosslinking step can be used to reach elastic modulus levels that are consistent with the target organ type for each individual organoid. For example, we can customize the liver bioinks to reach stiffnesses of 5-10 kPa, like a native liver, or cardiac bioinks (or microenvironment) to reach stiffnesses of 10-15 kPa like native cardiac tissue, in theory increasing the ability of these organoids to function in a similar manner to their native tissue counterparts.

The present invention is explained in greater detail in the drawings herein and the specification set forth below.

### BRIEF DESCRIPTION OF THE DRAWINGS

FIG. 1. Analysis of components present in tissue ECM-derived solutions for providing biochemical factors to hydrogel bioinks. A) A panel displaying the growth factors and cytokines amounts (pg/ml) measured in liver, cardiac, and skeletal muscle ECM solutions. B) The concentrations of collagen, GAGs, and elastin in liver, cardiac, and skeletal muscle ECM solutions.

FIG. 2. A) Strategy of formulation of printable bioinks comprised of acrylate-based crosslinkers (crosslinker 1), alkyne-based crosslinkers (crosslinker 2), thiolated HA, thiolated gelatin, and unmodified HA and gelatin. B) Implementation of bioprintable hydrogel bioinks. The bioink formulation is prepared and spontaneously crosslinks through thiol-acrylate binding, resulting in a soft, extrudable material. Bioprinting is performed. Lastly, the bioprinted structures are fused, stabilized, and brought to the target stiffness.

FIG. 3. Bioprinting testing of bioinks. A) A 7×7 mm pattern used for bioink deposition testing in the bioprinter. B) An initial formulation of a PEGDA and 4-arm PEG alkyne containing bioink after printing. C) Improved extrusion and end structure smoothness after addition of unmodified HA and gelatin to improve shear thinning and material smoothing.

FIG. 4. Bioink stiffness control and range of formulations. A) Demonstration of the capability to control bioink stiffness using Gel # 2 in this panel. After stage 1 crosslinking, the gel is relatively soft and able to be extruded smoothly. After stage 2 crosslinking by UV light, stiffness increases by more than an order of magnitude. B) A range of final stiffness levels of a variety of bioink formulations after stage 2 crosslinking, spanning from approximately 100 Pa to approximately 20 kPa.

FIG. 5. Design of biofabricated organoids.

FIG. 6. LIVE/DEAD imaging of organoids after extrusion biofabrication in compositions of the invention.

FIG. 7. Albumin and Urea analysis of organoids after extrusion biofabrication in compositions of the invention.

FIG. 8. Viability of acetaminophen (paracetamol; N-acetyl-p-aminophenol; “APAP”) treated organoids assessed by LIVE/DEAD staining.

FIG. 9. Albumin and Urea analysis of organoids after APAP treatment.

### DETAILED DESCRIPTION OF ILLUSTRATIVE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

### A. Compositions.

Compositions of the present invention may comprise live cells in a “bioink,” where the “bioink” is in turn comprised of a cross-linkable polymer, a post-deposition crosslinking group or agent; and other optional ingredients, including but not limited to growth factors, initiators (e.g., of cross-linking), water (to balance), etc. The compositions are preferably in the form of a hydrogel. Various components and properties of the compositions are discussed further below. Cells. Any type of cells, generally live cells, may be used to carry out the present invention, including but not limited to plant, animal, and microbial cells (e.g., yeast, bacteria, etc.). The cells may be combinations of multiple cell types, including combinations of cells from the same organism or species, symbiotic combinations of cells of different species, etc. In general the cells are preferably animal cells (e.g., bird, reptile, amphibian, etc.) and in some embodiments are preferably mammalian cells (e.g., dog, cat, mouse, rat, monkey, ape, human). The cells may be differentiated or undifferentiated cells, but are in some embodiments tissue cells (e.g., liver cells such as hepatocytes, pancreatic cells, cardiac muscle cells, skeletal muscle cells, etc.). Where tissue cells are employed, they may be incorporated as one cell type of multiple cell types for that tissue, and may be incorporated as discrete cells, or as cell aggregates such as organoids (which organoids may be unencapsulated or encapsulated; e.g., spheroids).

The cells may be incorporated into the composition in any suitable form, including as unencapsulated cells, or as cells previously encapsulated in spheroids Animal tissue cells encapsulated or contained in polymer spheroids can be produced in accordance with known techniques, or in some cases are commercially available (see, e.g., Insphero A G, 3D Hepg2 Liver Microtissue Spheroids (2012); Inspherio A G, 3D InSight™ IIuman Liver Microtissues, (2012)).

Cross-linkable prepolymers. Any suitable prepolymer can be used to carry out the present invention, so long as it can be further cross-linked to increase the elastic modulus thereof after deposition when employed in the methods described herein.

In some embodiments, the prepolymer is formed from the at least partial crosslinking reaction of: (i) an oligosaccharide (e.g., hyaluronic acid, collagen, combinations thereof and particularly thiol-substituted derivatives thereof) and (ii) a first crosslinking agent (e.g., a thiol-reactive crosslinking agent, such as polyalkylene glycol diacrylate, polyalkylene glycol methacrylate, etc., and particularly polyethylene glycol diacrylate, etc.; thiolated crosslinking agent to create thiol-thiol disulfide bonds; gold nanoparticles gold functionalized crosslinkers forming thiol-gold bonds; etc., including combinations thereof).

Cross-linking group. In some embodiments, the compositions include a post-deposition crosslinking group. Any suitable crosslinking groups can be used, including but not limited to multi-arm thiol-reactive crosslinking agent, such as polyethylene glycol dialkyne, other alkyne-functionalized groups; etc., including combinations thereof.

Initiators. Compositions of the invention may optionally, but in some embodiments preferably, include an initiator (e.g., a thermal or photoinitiator). Any suitable initiator that catalyzes the reaction between said prepolymer and the second (or post-deposition) crosslinking group (e.g., upon heating or upon exposure to light), may be employed.

Growth factors. Compositions of the invention may optionally, but in some embodiments preferably, include at least one growth factor (e.g., appropriate for the particular cells included, and/or for the particular tissue substitute being produced). An example is a decellularized extracellular matrix composition (“ECM”) from a tissue corresponding to the tissue cells (e.g., decellularized extracellular liver matrix when the live animal cells are liver cells; decellularized extracellular cardiac muscle matrix when the live animal cells are cardiac muscle cells; decellularized skeletal muscle matrix when the live animal cells are skeletal muscle cells; etc.). Additional collagens, glycosaminoglycans, and/or elastin (e.g., which may be added to supplement the extracellular matrix composition), etc., may also be included.

Elastic modulus. The composition preferably has an elastic modulus, at room temperature and atmospheric pressure, sufficiently low such that it can be manipulated and deposited on a substrate by whatever deposition method is employed (e.g., extrusion deposition). Further, the composition optionally, but in some embodiments preferably, has an elastic modulus, again at room temperature and atmospheric pressure, sufficiently high so that it will substantially retain the shape or configuration in which it is deposited until subsequent cross-linking (whether that cross-linking be spontaneous, thermal or photo-initiated, etc.). In some embodiments, the composition, prior to deposition, has a stiffness of from 0.05, 0.1 or 0.5 to 1, 5 or 10 kiloPascals, or more, at room temperature and atmospheric pressure.

### B. Methods.

The compositions of the invention may be used in any convenient manner In one non-limiting, but preferred, method of use, the compositions are used in a method of making a three-dimensional organ construct. Such a method generally comprises the steps of:

(a) providing a reservoir containing an extrudable hydrogel composition as described above, then

(b) depositing the hydrogel composition onto a substrate (e.g., by extrusion through a syringe); and then

(c) cross-linking said prepolymer with said second crosslinking group by an amount sufficient to increase the stiffness of said hydrogel and form said three-dimensional organ construct (e.g., by heating the hydrogel, irradiating the hydrogel composition with light (e.g., ambient light, UV light), altering the pH of the hydrogel; etc.).

The depositing step may be carried out with any suitable apparatus, including but not limited to that described in H.-W. Kang, S. J. Lee, A. Atala and J. J. Yoo, US Patent Application Pub. No. US 2012/0089238 (Apr. 12, 2012). In some embodiments, the depositing step is a patterned depositing step: That is, deposition is carried out so that the deposited composition is deposited in the forming of a regular or irregular pattern, such as a regular or irregular lattice, grid, spiral, etc.

In some embodiments, the cross-linking step increases the stiffness of said hydrogel by from 1 or 5 to 10, 20 or 50 kiloPascals, or more, at room temperature and atmospheric pressure.

In some embodiments, the hydrogel has a stiffness after said cross-linking step (c) of from 1 or 5 to 10, 20 or 50 kiloPascals at room temperature and atmospheric pressure.

In some embodiments, the method further comprises the step of depositing a supporting polymer (e.g., poly-L-lactic acid, poly(glycolic acid), polycaprolactone; polystyrene; polyethylene glycol, etc., including copolymers thereof such as poly(lactic-co-glycolic acid),) on said substrate in a position adjacent that of said hydrogel composition (e.g., concurrently with, after, or in alternating repetitions with, the step of depositing said hydrogel, and in some embodiments prior to the cross-linking step).

Any suitable substrate can be used for the deposition, including organic and inorganic substrates, and including substrates with or without features such as well, chambers, or channels formed thereon. For the particular products described below, the substrate may comprise a microfluidic device having at least one chamber (the chamber optionally but preferably associated with an inlet channel and/or an outlet channel), and the depositing is carried out in at least one chamber. In an alternative, the substrate may comprise a first planar member (e.g., a microscope cover slip), the depositing step may be carried out that planar member, and the method may further comprise the step of inserting that planar member into a chamber of a microfluidic device. Post-processing steps, such as sealing of chambers, and maintaining the viability of cells, may be carried out in accordance with known techniques.

### C. Products.

A variety of different products may be made with the methods and compositions described above. In a non-limiting, but preferred, example, the product may be a device useful for modeling animal tissue function (such as liver function) in vitro. Such a device may comprise: (a) a device body such as a microfluidic device having at least one chamber formed therein; (b) a hydrogel composition deposited in said chamber in a first pattern, (c) live animal tissue cells in said hydrogel composition; and (d) a structural support polymer deposited in said chamber adjacent said hydrogel. As noted above, the cells for such a device may comprise animal tissue cells, such as liver cells (e.g., hepatocytes), pancreatic cells, skeletal muscle cells; cardiac muscle cells, etc.).

The device body or microfluidic device may itself be formed of any suitable material or combination of materials. Examples include, but are not limited to, polydimethylsiloxane (PDMS), polystyrene, polymethyl methacrylate (PMMA), polyacrylamide, polyethylene glycol (PEG) including functionalized PEG (e.g. PEG diacrylate, PEG diacrylamide, PEG dimethacrylate, etc., or any of the foregoing PEGs in in multi-arm forms, etc.), natural polymers or proteins that can be cross-linked or cured (e.g., hyaluronic acid, gelatin, chondroitin sulfate, alginate, etc., including derivatives thereof that are functionalized with chemical groups to support cross linking, and including any of the “cross-linkable prepolymers” described above in cross-linked form, and combinations thereof. The device body may be formed by any suitable process, including molding, casting, additive manufacturing (3d printing), lithography, etc., including combinations thereof.

Where a structural support is included in the device as noted in the “Methods” section above, that structural support, like the hydrogel, may be patterned (e.g., a regular or irregular pattern, such as a regular or irregular lattice, grid, spiral, etc.).

In some embodiments, the tissue cells are contained in spheroids (e.g., polymer spheroids), which spheroids are contained in said hydrogel.

As noted above, the hydrogel is preferably cross-linked following deposition, such that the hydrogel residing in the device has a stiffness of from 1 or 5 to 10, 20 or 50 kiloPascals at room temperature and atmospheric pressure (e.g., preferably corresponding to the natural tissue in which the cells are found in vivo).

The device may be provided as a cartridge, or as a subcombination unit or “building block” configured in a manner suitable for “snap in” installation in a larger apparatus including pumps, detectors, or the like, as discussed further below.

### D. Packaging, Storage and Shipping.

Once produced, subcombination or “cartridge” devices as described above may be used immediately, or prepared for storage and/or transport.

To store and transport the product, a transient protective support media that is a flowable liquid at room temperature (e.g., 25° C.), but gels or solidifies at refrigerated temperatures (e.g., 4° C.), such as a gelatin mixed with water, is added into the device to substantially or completely fill the chamber(s), and preferably also any associated conduits.

Any inlet and outlet ports are capped with a suitable capping element (e.g., a plug) or capping material (e.g., wax). The device is then packaged together with a cooling element (e.g., ice, dry ice, a thermoelectric chiller, etc.) and all placed in a (preferably insulated) package.

Alternatively, to store and transport the product, a transient protective support media that is a flowable liquid at cooled temperature (e.g., 4° C.), but gels or solidifies at warmed temperatures such as room temperature (e.g., 20° C.) or body temperature (e.g., 37° C.), such as poly(N-isopropylacrylamide and poly(ethylene glycol) block co-polymers.

Upon receipt, the end user simply removes the device from the associated package and cooling element, allows the temperature to rise or fall (depending on the choice of transient protective support media), uncaps any ports, and removes the transient protective support media with a syringe (e.g., by flushing with growth media).

The present invention is explained in greater detail in the following non-limiting Examples.

### EXAMPLES

### Materials and Methods

Materials. Hydrochloric acid (HCl) was from Fischer Scientific (Houston, Tex.). Pepsin (porcine gastic mucosa) was from Sigma Aldrich (St. Louis, Mo., USA). Heprasil (est. 160 kDa MW), Gelin-S, and Extralink (PEGDA, 3.4 kDa MW) were used from HyStem-HP hydrogel kits from ESI-BIO (Alameda, Calif., USA). PEG 4-Arm Acrylate (10 and 20 kDa MW), PEG 4-Arm Alkyne (10 kDa MW), and PEG 8-Arm Alkyne (10 kDa MW) were from Creative PEGWorks (Winston-Salem, N.C., USA).

Preparation of tissue-specific extracellular matrix (ECM) digest. Tissue-specific

ECM digest solutions were prepared as previously described for liver (A. Skardal, L. Smith, S. Bharadwaj, A. Atala, S. Soker and Y. Zhang, Biomaterials, 33, 4565 (2012).). Fresh liver, cardiac, or skeletal muscle tissue was rinsed with chilled Dulbecco's phosphate buffered saline (DPBS). The tissues were cut into 10 cm by 0.5-1.0 cm strips and minced with surgical scalpels. Minced tissue was transferred to 500 ml distilled water and shook on a rotary shaker at 200 rpm for 3 days at 4° C., during which the water was changed three times per day. The tissues were treated with 2% Triton X-100 for 4 days followed by 2% TX-100 with 0.1% NH4OH for 24 h. During the TX-100 rinses, solutions were changed twice daily. The decellularized tissues were washed for 2 additional days in distilled water to remove any traces of TX-100, after which they were stored at 4° C. until further use.

Decellularized tissue ECMs were frozen and lyophilized for 48 h. Following lyophilization, samples were ground into a powder with a freezer mill. One gram of liver tissue or liver ECM powder was mixed with 100 mg Pepsin (Porcine gastric mucosa, 3400 units of protein, Fisher Scientific, Fair Lawn, N.J.) and sterilized by gamma irradiation (1 Mrad). All subsequent procedures following sterilization were carried out under sterile conditions. Hydrochloric acid (0.1 N, 100 mL) was added to the sterilized materials and incubated for 48 h at room temperature. The resulting mixture was transferred to a 50 ml conical tube and centrifuged at 3000 rpm for 15 min. The supernatant was removed and the pellet was discarded. This was repeated 3 times until the supernatant was clear. To ensure there was no more particulate matter remaining, the suspension was filtered through a 0.45 mm syringe filter (Fisher Scientific). The resulting decellularized ECM solutions were stored at 80° C. until further use.

Hydrogel bioink formulations and preparation. Prior to hydrogel formulation, a photonitiator, Irgacure 2959 (4-(2-hydroxyethoxy)phenyl-(2-propyl)ketone, Sigma), was dissolved in water at 0.05% w/v. To form hydrogel bioinks, first the base material components from HyStem-HP hydrogel kits (ESI-BIO, Alameda, Calif.) were dissolved in the water-phoinitiator solution. Briefly, Heprasil and Gelin-S were dissolved in water-phoinitiator solution to make 2% w/v solutions. Extralink, the crosslinker, was dissolved in water-phoinitiator solution to make a 4% and 8% w/v solution. Additionally, multi-arm PEG-based crosslinkers were prepared separately: PEG 4-Arm Acrylate (10 kDa or 20 kDa MW; 4% and 8% w/v), PEG 4-Arm Alkyne (10 kDa MW; 4% w/v), and PEG 8-Arm Alkyne (10 kDa MW; 8%, 10%, 16% and 20% w/v).

Following dissolution of all materials, hydrogels were formulated by 2 general schemes. In the first, 4 parts 2% Heprasil, 4 parts 2% Gelin-S, 1 part crosslinker 1, 1 part crosslinker 2 is combined with 10 parts tissue ECM solution (or water as a generic non-tissue-specific hydrogel). The resulting mixture is vortexed to mix prior to use. For extrusion or bioprinting testing, the mixture is transferred into a syringe or printer cartridge and allowed to crosslink spontaneously for 30 minutes (stage 1 crosslinking). For rheological measurements, the mixture is transferred into a 35 mm petri dish and allowed to crosslink. In the second formulation approach, the Heprasil, Gelin-S, and crosslinker solution is not further diluted with tissue ECM solution or water in order to achieve an increased polymer concentration. Instead, the photoinitiator is dissolved in the tissue ECM solutions at 0.05% w/v, which subsequently is used to dissolve the Heprasil, Gelin-S, and various crosslinkers. These components are then combined in the same 4:4:1:1 volume ratio. The materials were transferred into syringes, printer cartridges or petri dishes and allowed to spontaneously crosslink (stage 1 crosslinking) as described above for implementation. For secondary crosslinking (stage 2) the stage 1-crosslinked gels are irradiated with ultraviolet light (365 nm, 18 w/cm²) to initiate a thiol-alkyne polymerization reaction.

Printer compatibility testing. Extrusion-based bioprinting was tested first on the laboratory bench with simple extrusion tests using standard syringes and small gauge needle tips (20-30 gauge). Next, bioink preparations were loaded into printer cartridges, allowed to undergo spontaneous stage 1 crosslinking, and extrusion compatibility for bioprinting was assessed using a custom 3-D bioprinting device designed in house specifically for tissue construct printing (See, e.g., H. Kang, S. Lee, A. Atala and J. Yoo, US Patent Application Pub. No. US 2012/0089238 (Apr. 12, 2012)). A 7×7 mm pattern was implemented for testing purposes. To improve shear thinning and extrusion properties, unmodified HA and gelatin was supplemented to the bioinks (1.5 mg/mL and 30 mg/mL) (Sigma). The tendency for the bioink to be extruded smoothly versus in irregular clumps was observed.

Determination of bioink mechanical properties by rheology. For determination of bioink mechanical properties, hydrogels were prepared as described above and pipetted into 35 mm petri dishes where they underwent the stage 1 spontaneous crosslinking. Rheological testing was performed using an HR-2 Discovery Rheometer (TA Instruments, Newcastle, Del.). A 12-mm steel disc was lowered until contact with the surface of the hydrogel was made. The disc was lowered further until the axial force on the instrument, or normal force acting on the disc from the hydrogel, equaled 0.4 N. At this point G′ was measured for each hydrogel using a shear stress sweep test ranging from 0.6 to 10 Pa at an oscillation frequency of 1 Hz applied by the rheometer.

For determination of the stiffness after completion of the second stage crosslinking, identical untested hydrogels were further crosslinked by UV photopolymerization after which G′ measurement was performed as described.

Bioprinting of liver organoids for biological validation of bioinks. Primary liver hepatocyte-based spheroids were formed by hanging drop method. Spheroids were harvested and suspended within a liver-specific bioink formulation containing liver ECM materials, drawn into a syringe compatible with the bioprinting, and the bioink was allowed to spontaneously crosslink through thiol-acrylate bonding. After 30 minutes, the spheroid-containing bioink was bioprinted within a polycaprolactone support pattern on a plastic coverslip. Following bioink depostion, UV light was used to initiate the second crosslinking step, stabilizing the bioink further and raising the bioink stiffness to a value similar to native liver, thus comprising the larger liver organoid. As a control, a gelatin-based hydrogel previously used in the bioprinter was used to bioprint spheroids in parallel. Following printing, viability of the organoids was assessed using a LIVE/DEAD stain, after which the organoids were fixed in paraformaldehyde, and imaged with a Leica TCS LSI macro-confocal microscope to determine the relative amounts of viable (green fluorescent) and dead (red fluorescent) cells.

Functional analysis of liver organoids and toxic insult. Organoids were prepared as described above. 9 organoids were placed in microreactors for 14 day culture time courses, during which media aliquots would be sampled and reserved for functional analysis. Microreactors consist of polydimethylsiloxane (PDMS) devices with chambers for organoid placement, and channels through which cell culture media can be circulated from a reservoir using a micro-peristaltic pump. After sampling media aliquots on day 3 and day 6 for baseline functional metrics, 3 organoids continued in culture with normal media; 3 organoids were administered media containing 1 mM acetaminophen, and 3 organoids were administered 10 mM acetaminophen in media. Media aliquots were collected on days 10 and 14, after which urea and albumin secretion were quantified and viability was assessed by LIVE/DEAD imaging.

Toxic insult and clinical relevant intervention with N-acetyl-L-cysteine. Organoids were prepared again as described above and placed in microreactor devices for 14 days of culture. After sampling media aliquots on day 3 and day 6 for baseline functional metrics, 1 group of organoids continued in culture with normal media; another group of organoids was administered media containing 10 mM acetaminophen, and the third group of organoids was administered 10 mM acetaminophen plus 20 mM N-acetyl-L-cysteine (NAC) in media. Media aliquots were collected on day 10 and 14, after which urea and albumin secretion were quantified

### Results: Characterization.

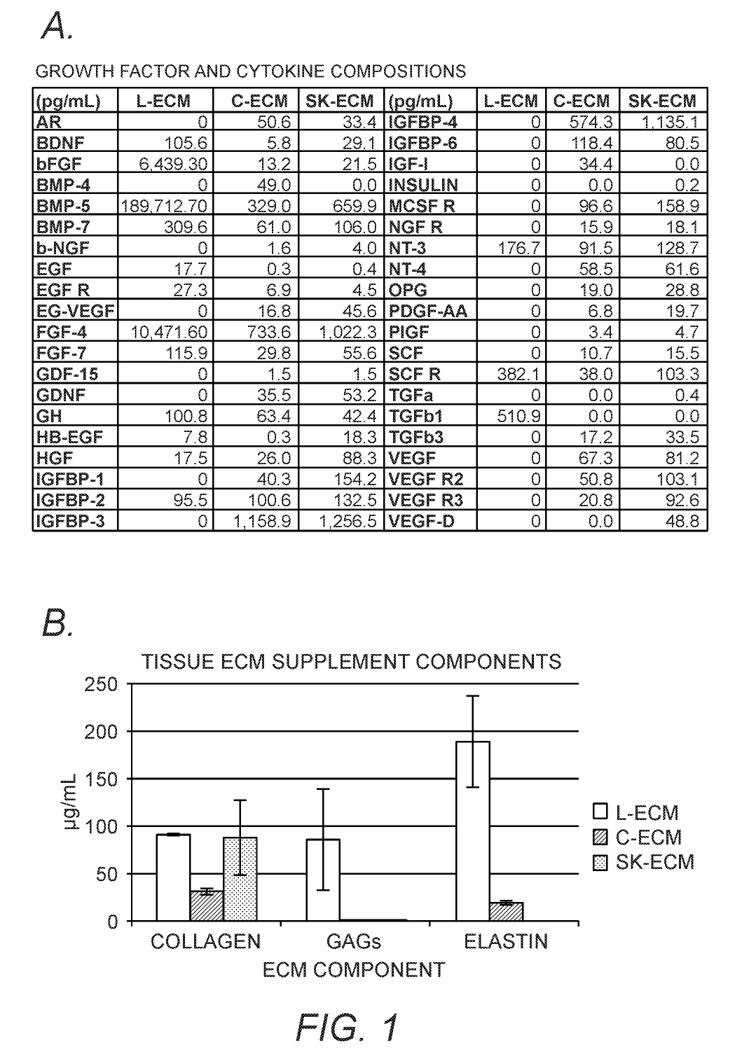

ECM component analysis. Liver ECM solutions were analyzed previously by a series of colorimetric assays (A. Skardal et al., supra). Two formulations were analyzed: 1) LEE, decellularized liver prepared as described above; and LTE, fresh liver tissue that was prepared identically, with the exception that it was not decellularized. The results revealed a clear trend, in which LEE solutions contained greater concentrations of collagen, glycosaminoglycans (GAGs), and elastin (FIG. 1A). Specifically, the total collagen content of LEE, 91.33 mg/mL, was significantly greater than that of LTE, which was 4.17 mg/mL (p<0.001), the elastin content of LEE, 189.33 mg/mL, was significantly greater than that of LTE, which was 36.00 mg/mL (p<0.05) and the GAG content of LEE, 86.00 mg/mL, was greater than that of LTE, which was 40.67 mg/mL, but not significantly (p >0.05).

Cardiac and skeletal muscle ECM solutions (both decellularized preparations) were assessed in the same manner.

Growth factor analysis. Liver ECM solutions were analyzed previously by the Quantibody Growth Factor Array, which revealed that, in general, LEE contained higher concentrations of growth factors and cytokines (shown in pg/mL, FIG. 1B). Of particular interest was that brain-derived neurotrophic factor (BDNF), bFGF, bone morphogenetic protein 5 (BMP-5), FGF-4, insulin-like growth factor binding protein 2 (IGFBP-2), and TGF-b1 were relatively conserved between both LEE and LTE. However, LEE also contained BMP-7, EGF, FGF-7, growth hormone (GH), heparin-binding EGF-like growth factor (HB-EGF), HGF and neurotrophin 3 (NT-3), which were not observed or were negligible in LTE. On the other hand, BMP-4, and glial-derived neurotrophic factor (GDNF) were present in LTE, but not in LEE (A Skardal et al., supra). Cardiac and skeletal muscle ECM solutions (both decellularized preparations) were assessed in the same manner.

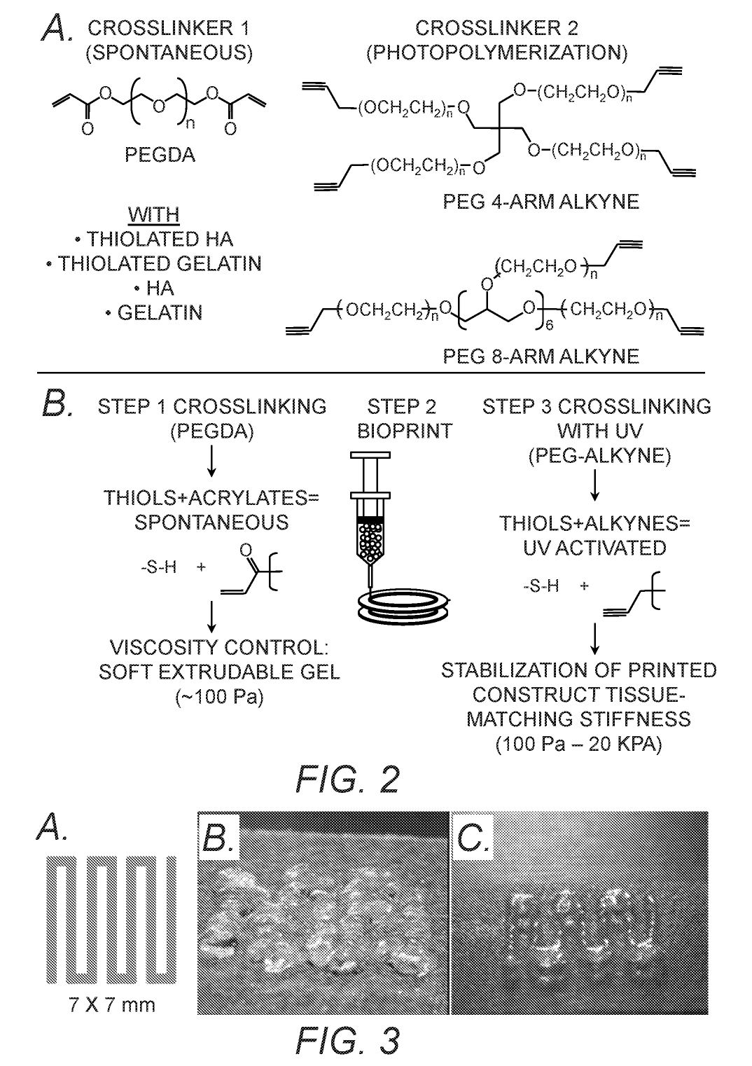

Hydrogel bioink preparation and extrusion bioprinting testing. Strategy and implementation of stage 1 and stage 2 crosslinking of the hydrogel bioinks is described in FIG. 2A and B. A 7'7 mm pattern was implemented for testing purposes (FIG. 3A). Initial tests showed that the initial formulations were extrudable, but appeared irregular and clumped during and after extrusion (FIG. 3B). To improve shear thinning and extrusion properties, unmodified HA and gelatin was supplemented to the bioinks (1.5 mg/mL and 30 mg/mL). The improved smooth printed structure is shown in FIG. 3C.

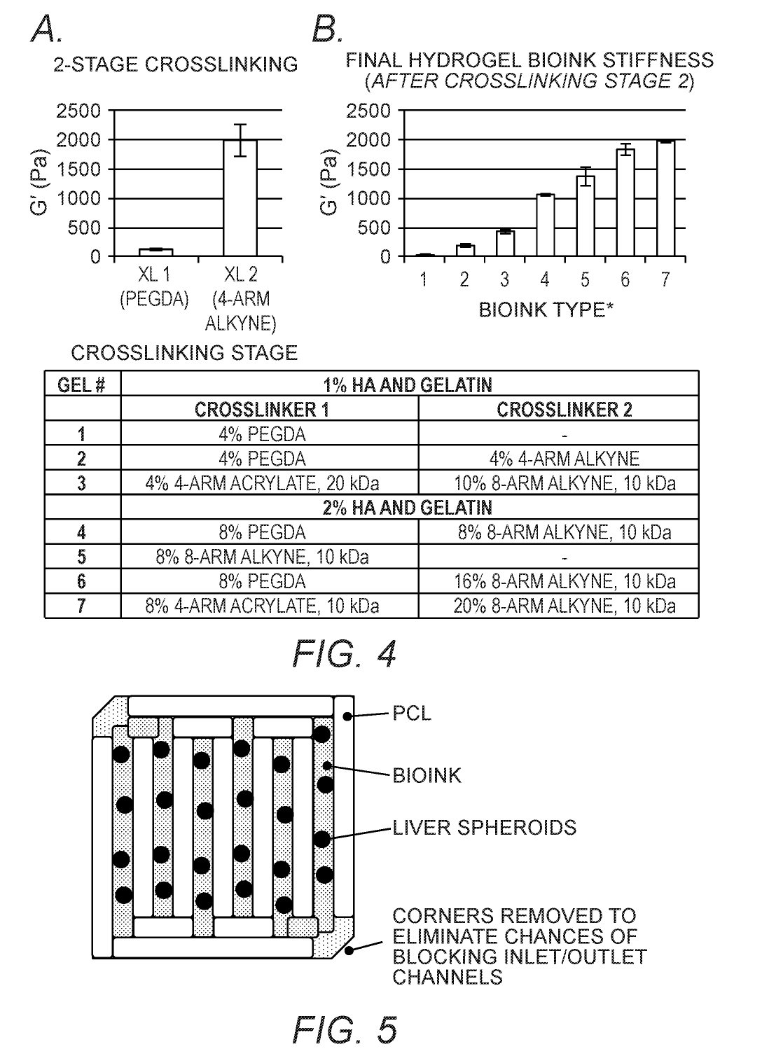

Rheological testing. As described in the methods, hydrogels of different formulations were prepared for rheological assessment of their mechanical properties. FIG. 4A shows the increase in shear elastic modulus (G′) in a gel that after spontaneous crosslinking with PEGDA had a G′ of 113.66 Pa. This is the stage during which the hydrogel can be extruded as a bioink. After UV crosslinking with a 4-anti PEG Alkyne crosslinker, the G′ value increases to 1981.79 Pa. FIG. 4B shows the range of G′ values that can be achieved through the secondary Alkyne-based crosslinking step, allowing mimicry of many tissue types in the body. Table 1 shows a range of hydrogel stiffness within the range of this system, formulations, and associated tissue types.

TABLE 1
A) Formulations for creating liver, heart, and skeletal muscle-specific hydrogel
bioinks. B) Formulations for additional tissue types of interest.

| | | | Physical Parameters | | | | |
|---|---|---|---|---|---|---|---|
| | | Biochemical | | Biofabrication | | | |
| | Tissue | Parameters | Crosslinker 1 | G′ | Crosslinker 2 | | |
| | | | | | | Endpoint G′ | |
| A | Liver | GFs/ECM from | PEGDA | 100-200 Pa | 8-Arm PEG | ~10 | kPa |
| | | dissolve/decell | | | Alkyne | | |
| | | liver tissue | | | | | |
| | Heart | GFs/ECM from | PEGDA | 100-200 Pa | 8-Arm PEG | ~10-15 | kPa |
| | | dissolve/decell | | | Alkyne | | |
| | | cardiac tissue | | | | | |
| | Skeletal Muscle | GFs/ECM from | 4-Arm PEG | 200-400 Pa | 8-Arm PEG | ~15-20 | kPa |
| | | dissolve/decell | Acrylate | | Alkyne | | |
| | | skeletal muscle | | | | | |
| | | tissue | | | | | |
| | | | | | | Target | |
| | | | | | | Endpoint G′ | |
| B | Bone Marrow | Bone marrow ECM | PEGDA | 100-200 Pa | PEG-Di-Alkyne/ | ~1 | kPa |
| | | | | | 4-Arm PEG | | |
| | | | | | Alkyne Blend | | |
| | Fat | Fat ECM | PEGDA | 100-200 Pa | PEG-Di-Alkyne/ | ~1 | kPa |
| | | | | | 4-Arm PEG | | |
| | | | | | Alkyne Blend | | |
| | Brain/Nerve | Brain/nervous | PEGDA | 100-200 Pa | 4-Arm PEG | ~1-2 | kPa |
| | | tissue ECM | | | Alkyne | | |
| | Lung | Lung ECM | PEGDA | 100-200 Pa | 4-Arm PEG | ~3-5 | Kpa |
| | | | | | Alkyne | | |
| | Kidney | Kidney ECM | PEGDA | 100-200 Pa | 8-Arm PEG | ~10 | kPa |
| | | | | | Alkyne | | |
| | Smooth Muscle | Smooth muscle | 4-Arm PEG | 200-400 Pa | 8-Arm PEG | ~10-15 | kPa |
| | | ECM | Acrylate | | Alkyne | | |
| | Cartilage/ | Cartilage/Tendon | 4-Arm PEG | 200-400 Pa | ? | 100-1000 | kPa |
| | Tendon | ECM | Acrylate | | | | |

### Results: Validation.

Bioink maintenance of primary organoid viability in a bioprinting biofabrication setting. The biofabricated organoids were designed as depicted in FIG. 5. Using the integrated printing approach of printing both polycaprolactone (PCL) and hydrogels, a PCL channel structure was printed inside of which the bioink with the liver cells was printed. The channel structures provide stability to the hydrogel when under flow as well as increasing the height-width aspect ratio of the hydrogel and cells. These structures were printed on plastic coverslips that were customized to fit inside the microfluidic microreactor chambers. These square coverslips feature 2 additional cuts in the corners to prevent occlusion of the micro-channels providing the inlet and outlet flows to the organoid chamber in the microreactors.

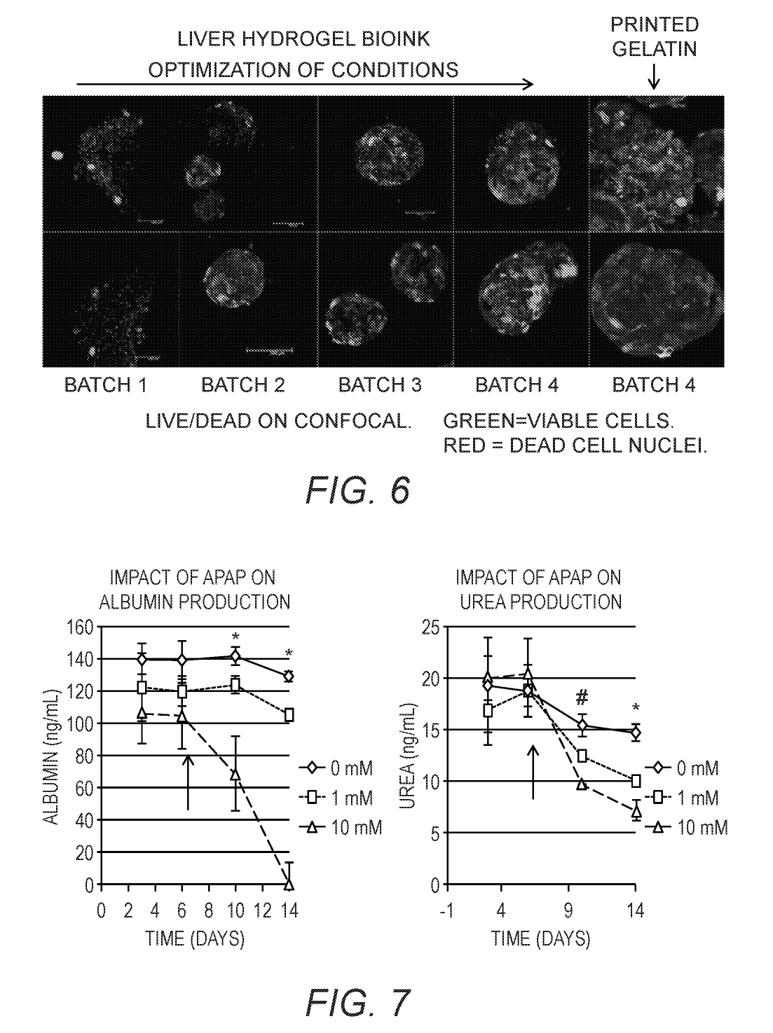

Most spherical organoids within the overall construct stayed spherical during the printing process and maintained their original shape in culture. In earlier batches without PCL channels, some organoids were observed to become disfigured, compressed, or even torn during the printing process. This indicates a substantial improvement in the biofabrication technique. Uniformity of spherical organoid distribution and quantity was improved. In this batch, each organoid construct that was moved to microreactor culture (n=9) contained between 40-45 spherical organoids. In past batches this number varied between 10 and 30. Multiple batches of organoids were biofabricated allowing opimization of the biofabrication conditions. Temperature was adjusted to remain near 37° C. in the bioink and in the bioprinting chamber. Biofabrication preparation and methodology was performed in less time. Hepatocyte culture medium was added to the bioink to provide nutrients to the cells during printing. LIVE/DEAD imaging shows the increased viability of multiple iterations of the organoids after extrusion biofabrication in the bioink (FIG. 6). A gelatin control was used in parallel with Batch 4 as a comparison. Simple gelatin gels are commonly used for extrusion biofabrication.

Ability to support primary cell function in vitro using liver bioinks: Baseline secretory activity and toxic insult. Liver organoids were prepared and biofabricated as described above, placed in microfluidic microreactors and cultured for 14 days. On day 6, organoids received normal media, or 1 of 2 concentrations of APAP.

Albumin analysis (FIG. 7) by a Human Albumin ELISA Kit (Alpha Diagnostic International) revealed constant albumin production by bioprinted liver organoids through day 6, remaining on average near 120 ng/mL. It should be noted that during these 2 baseline timepoints we observed a trend in secretion magnitude, with 0 mM organoids secreting the most, followed by the 1 mM orgnanoid group, and then the 10 mM group. This decrease in baseline albumin production is believed to be due to the time at which the organoids were printed. The organoids in group 1 were printed first, and therefore have a slightly improved viability, which translates into improved albumin secretion. Despite this trend, albumin levels at these 2 time points were not statistically significant in comparison to one another. But in future studies, organoids will be randomly assigned to experimental treatment groups. Following APAP administration after day 6, albumin levels were significantly decreased in both the 1 mM and 10 mM groups compared to the 0 mM control (p<0.05). Additionally, the 10 mM group albumin levels were significantly decreased compared to the 1 mM group (p<0.01). In fact, at day 14 the albumin levels in the 10 mM group were nearly immeasurable.

Urea analysis (FIG. 7) by a QuantiChrom Urea Colorimetric Assay Kit (BioAssay Systems) showed less drastic results than the albumin analysis, yet the results were still significant statistically with expected trends. Urea levels were not significantly different between the 3 groups during the time points prior to APAP administration. After APAP administration, measured urea levels appeared to drop in a dose dependent manner with respect to APAP concentration. On the day 10 time point, the 0 mM control group albumin level was significantly higher than both the 1 mM and 10 mM group (p<0.05). On the day 14 time point, all 3 groups were significantly different from one another (p<0.05)

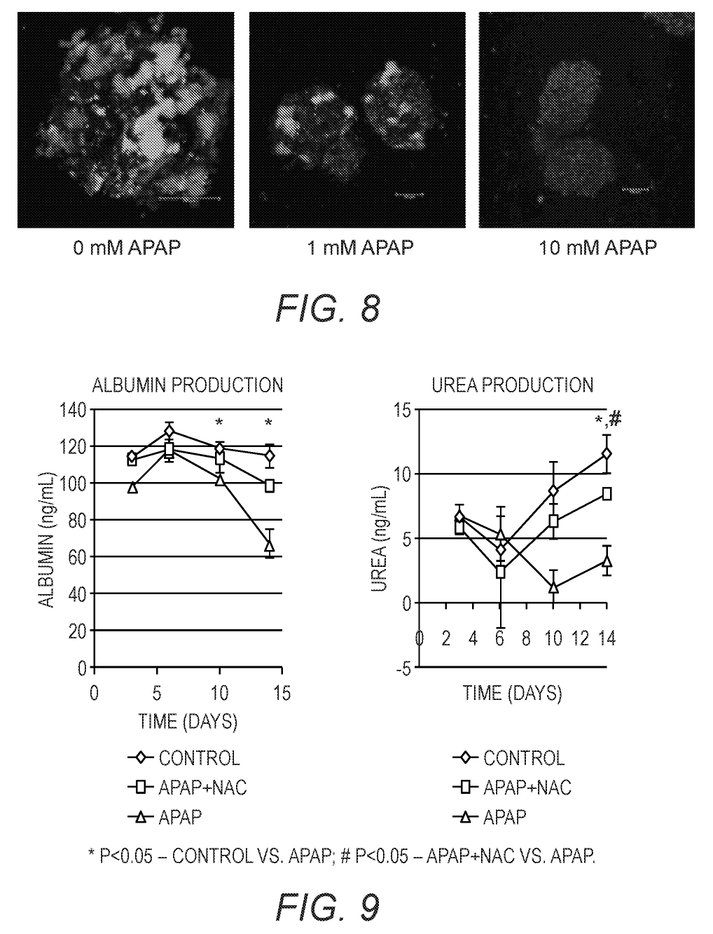

Viability of the 0 mM, 1 mM, and 10 mM APAP treated organoids was assessed by LIVE/DEAD staining and imaging using the macro-confocal microscope as has been described before (FIG. 8). Based on the ratio of live cells to dead cells, it was evident that the 0 mM control group maintained a relatively high level of viability (70-90% at day 14) throughout the 14 day experiment. In comparison, the 1 mM group had decreased viability (30-50% at day 14), while the 0 mM group appeared to have nearly no viable cells at day 14.

APAP toxicity testing and N-acetyl-L-cysteine intervention. As described previously, liver organoids will prepared and bioprinted as described in previous reports. These organoids were used to set baseline functional metrics by media aliquots reserved on day 3 and day 6. Organoids would then undergo toxic insult by acetaminophen (10 mM), but some groups would also be administered N-acetyl-L-cysteine (20 mM) as a clinically relevant counteimeasure. Media aliquots were reserved for functional analysis on days 3, 6, 10, and 14.

Albumin analysis (FIG. 9) by a Human Albumin ELISA Kit (Alpha Diagnostic International) revealed constant albumin production by bioprinted liver organoids through day 6, remaining on average near 120 ng/mL, consistent with the experiment reported previously in the June report. Following administration of APAP only, we observed a decrease in detected albumin. This decrease was statistically significant (p<0.5) compared to the untreated control organoids at day 10 and day 15. The co-administration of APAP and NAC saw a slight decrease in detected albumin production, decreasing to 98 ng/mL by day 14, however, this value was not significantly different that the control organoids nor the APAP only organoids. The general trend of the data was appropriate, suggesting that the liver organoids respond to APAP correctly, and can be rescued by NAC, as patients in the clinic might be.

Urea analysis (FIG. 9) by a QuantiChrom Urea Colorimetric Assay Kit (BioAssay Systems) also showed results with promising trends. Following APAP administration, detected urea decreased as expected. In the control organoids, as well as the APAP+NAC organoids, detected urea production increased over time. There was no statistical significance between groups on day 3 and day 6 (to be expected), nor on day 10, despite the drop in APAP urea production. However, on day 14, both the control organoids and APAP+NAC organoids had increased detected urea levels (p<0.05). As with the albumin data, these trends are appropriate and expected.

The foregoing is illustrative of the present invention, and is not to be construed as limiting thereof. The invention is defined by the following claims, with equivalents of the claims to be included therein.

1. An extrudable hydrogel composition useful for making a three-dimensional organ construct, comprising:
(a) a cross-linkable prepolymer;
(b) a post-deposition crosslinking group;
(c) optionally, but in some embodiments preferably, an initiator that catalyzes the reaction between said prepolymer and said post-deposition crosslinking group;
(d) live cells (e.g., plant, animal, or microbial cells); and
(e) optionally, but in some embodiments preferably, at least one growth factor; and
(f) optionally, water to balance.

2. The composition of claim 1, wherein said at least one growth factor comprises a decellularized extracellular matrix composition.

3. The composition of claim 1, wherein said initiator is present and comprises a thermal initiator or photoinitiator.

4. The composition of claims 1, wherein said post-deposition crosslinking group comprises a multi-arm thiol-reactive crosslinking agent.

5. The composition of claims 1, wherein said prepolymer is formed from the at least partial crosslinking reaction of: (i) an oligosaccharide and (ii) a first crosslinking agent.

6. The composition of claims 1, wherein said composition is viscous.

7. The composition of claims 1, wherein said cells are animal tissue cells.

8. The composition of claims 1, wherein said cells are encapsulated in spheroids.

9. A method of making a three-dimensional organ construct, comprising the steps of:
(a) providing a reservoir containing an extrudable hydrogel composition, said composition comprising:
(i) a cross-linkable prepolymer
(ii) a post-deposition crosslinking group;
(iii) optionally, an initiator that catalyzes the reaction between said prepolymer and said post-deposition crosslinking group;
(iv) live cells; and
(v) optionally, but in some embodiments preferably, decellularized extracellular matrix from a tissue corresponding to said tissue cells; and
(vi) optionally, water to balance; then
(b) depositing said hydrogel composition onto a substrate; and then
(c) cross-linking said prepolymer with said post-deposition crosslinking group by an amount sufficient to increase the stiffness of said hydrogel and form said three-dimensional organ construct.

10. The method of claim 9, wherein said crosslinking step is a thermally initiated or photoinitiated crosslinking step.

11. The method of claim 9, wherein said prepolymer is formed from the at least partial crosslinking reaction of: (i) an oligosaccharide and (ii) a first crosslinking agent.

12. The method of claims 9, wherein said hydrogel composition is sufficiently stiff to retain a configuration of deposition on said substrate from said depositing step to said cross-linking step.

13. The method of claim 9, wherein:
(i) said hydrogel has a stiffness prior to said depositing step of from 0.05, 0.1 or 0.5 to 1, 5 or 10 kiloPascals, or more, at room temperature and atmospheric pressure; and/or
(ii) said cross-linking step increases the stiffness of said hydrogel by from 1 or 5 to 10, 20 or 50 kiloPascals, or more, at room temperature and atmospheric pressure; and/or
(ii) said hydrogel has a stiffness after said cross-linking step (c) of from 1 or 5 to 10, 20 or 50 kiloPascals at room temperature and atmospheric pressure.

14. The method of claims 9, wherein said depositing step is a patterned deposition step.

15. The method of claims 9, further comprising the step of:
(d) depositing a supporting polymer on said substrate in a position adjacent that of said hydrogel composition.

16. The method of claims 9, wherein:
said substrate comprises a microfluidic device having at least one chamber, and said depositing is carried out in said at least one chamber; or
said substrate comprises a first planar member, said depositing step is carried out on said planar member, and said method further _{\}comprises the step of inserting said planar member into a chamber of a microfluidic device.

17. A device useful for modeling cellular function in vitro, comprising:
(a) a microfluidic device substrate having at least one chamber formed therein;
(b) a hydrogel composition deposited in said chamber in a first pattern,
(c) live cells in said hydrogel composition; and
(d) a structural support polymer deposited in said chamber adjacent said hydrogel.

18. The device of claim 17, wherein said cells are animal cells.

19. The device of claim 18, wherein said cells are liver tissue cells, pancreatic cells, skeletal muscle cells, cardiac muscle cells, or a combination thereof.

20. The device of claims 17, wherein said structural support is patterned.

21. The device of claims 17, wherein said organ tissue cells are contained in spheroids, which spheroids are contained in said hydrogel.

22. The device of claims 17, wherein said hydrogel has a stiffness of from 1 or 5 to 10, 20 or 50 kiloPascals at room temperature and atmospheric pressure.

23. The device of claims 17, packaged in a container with a transient protective support media in said chamber in gelled form, and optionally together with a cooling element in said container.
